(19) United States
(12) Patent Application Publication
(10) Pub. No.: US 2025/0000765 A1
(43) Pub. Date: Jan. 2, 2025

(54) COMPOSITION FOR CLEANSING THE HAIR

(71) Applicant: L'OREAL, Paris (FR)

(72) Inventors: Jie DENG, Shanghai (CN); Qing YANG, Shanghai (CN); Shichen ZHANG, Shanghai (CN)

(73) Assignee: L'OREAL, Paris (FR)

(21) Appl. No.: 18/710,925

(22) PCT Filed: Nov. 30, 2021
(86) PCT No.: PCT/CN2021/134373

§ 371 Date: May 16, 2024

(51) Int. Cl. A61K 8/365; A61K 8/41; A61K 8/73; A61Q 5/02

(52) U.S. Cl. CPC A61K 8/365; A61K 8/41; A61K 8/737; A61Q 5/02; A61K 2800/30

(57) ABSTRACT

It relates to a composition for cleansing the hair comprising: a) at least one anionic surfactant; and b) at least one alpha hydroxyl acid and at least one beta hydroxyl acid. It also relates to a process for cleansing the hair comprising applying the composition onto the hair, and then rinsing the hair with water after an optional period of exposure.

### TECHNICAL FIELD

The present invention relates to a composition for cleansing the hair. The present invention also relates to a process for cleansing the hair.

### BACKGROUND ART

Greasy scalp and hair are becoming the annoying scalp issue worldwide. People usually use oil control shampoo to remove dirt and sebum on scalp.

There are many shampoos developed to cleanse the hair and the scalp.

For example, U.S. Pat. No. 9,018,150B1 discloses a cleansing composition containing from about 6% to about 20% of at least one nonionic surfactant; from about 3% to about 10% of at least one amphoteric surfactant; from about 2% to about 8% of at least one anionic surfactant; and from about 0.1% to about 5% of at least one cationic conditioning surfactant, cationic conditioning amine, or a combination thereof; wherein the amount of nonionic surfactant present in the final composition is greater than the amount of the amphoteric surfactant, and the ratio of the nonionic surfactant (a) to anionic surfactant (c) is at least about 1.9 as much as the anionic surfactant, based on the weight percent of each surfactant in the final composition.

However, for most commercial shampoos available, consumers are not satisfied with the long-lasting clean between two hair wash. Most commercial shampoos available only contains surfactants as cleansing agents, it can only remove liquid sebum just secreted. Liquid sebum composition will be transferred by fungi and bacterial on scalp and form stubborn sebum together with dead cells. Most shampoos can hardly remove the stubborn sebum so that they are not efficient for clean greasy scalp.

Thus, it is desired that the shampoo can keep the hair and the scalp clean for longer time.

Therefore, there is a need to develop compositions for cleansing the hair, which can deliver long lasting cleaning effect.

### SUMMARY OF THE INVENTION

An object of the present invention is thus to develop compositions for cleansing the hair, which can deliver long lasting cleaning effect.

Another object of the present invention is to provide a process for cleansing the hair.

Thus, according to a first aspect, the present invention provides a composition for cleansing the hair comprising:
a) at least one anionic surfactant; and
b) at least one alpha hydroxy acid and at least one beta hydroxy acid.

Preferably, the composition according to the present invention is silicon free.

According to a second aspect, the present invention provides a process for cleansing the hair comprising applying the composition as described above onto the hair, and then rinsing the hair with water after an optional period of exposure.

The inventors have found that the composition according to the present invention can provide good cleaning effect, i.e., the composition can keep the hair clean for at least 24 hours.

Other subjects and characteristics, aspects and advantages of the present invention will emerge even more clearly on reading the detailed description and the examples that follow.

### DETAILED DESCRIPTION OF THE INVENTION

As used herein, unless otherwise indicated, the limits of a range of values are included within this range, in particular in the expressions “between . . . and . . . ” and “from . . . to . . . ”.

As used herein, the term “comprising” is to be interpreted as encompassing all specifically mentioned features as well as optional, additional, unspecified ones.

As used herein, the use of the term “comprising” also discloses the embodiment wherein no features other than the specifically mentioned features are present (i.e. “consisting of”).

Unless otherwise defined, all technical and scientific terms used herein have the same meaning as commonly understood by those skilled in the field the present invention belongs to. When the definition of a term in the present description conflicts with the meaning as commonly understood by those skilled in the field the present invention belongs to, the definition described herein shall apply.

Unless otherwise specified, all numerical values expressing amount of ingredients and the like used in the description and claims are to be understood as being modified by the term “about”. Accordingly, unless indicated to the contrary, the numerical values and parameters described herein are approximate values which are capable of being changed according to the desired performance obtained as required.

As used herein, the expression “at least one” used in the present description is equivalent to the expression “one or more”.

As used herein, “silicon free” means that no compound containing silicon is added on purpose and the content of the compound containing silicon in the composition is less than 0.5 wt. %, particularly less than 0.1 wt. %, relative to the total weight of the composition. In particular, there is no silicon in the composition.

According to the first aspect of the present invention, a composition for cleansing the hair comprises:
a) at least one anionic surfactant; and
b) at least one alpha hydroxy acid and at least one beta hydroxy acid.

### Anionic Surfactants

According to the first aspect of the present invention, the composition comprises at least one anionic surfactant.

The term “anionic surfactant” means a surfactant comprising, as ionic or ionizable groups, only anionic groups.

In the present description, a species is termed as being “anionic” when it bears at least one permanent negative charge or when it can be ionized as a negatively charged species, under the conditions of use of the composition of the invention (for example the medium or the pH) and not comprising any cationic charge.

The anionic surfactants may be sulfate, and/or sulfonate surfactants. It is understood in the present description that:
the sulfonate anionic surfactants comprise at least one sulfonate function (—SO₃H or —SO₃) and may optionally also comprise one or more sulfate functions, but do not comprise any carboxylate functions; and
the sulfate anionic surfactants comprise at least one sulfate function but do not comprise any carboxylate or sulfonate functions.

The sulfonate anionic surfactants that may be used comprise at least one sulfonate function (—SO₃H or —SO₃).

They may be selected from the following compounds: alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds;

the alkyl groups of these compounds comprising from 6 to 30 carbon atoms, especially from 12 to 28, better still from 14 to 24 or even from 16 to 22 carbon atoms; the aryl group preferably denoting a phenyl or benzyl group;

these compounds possibly being polyoxyalkylenated, especially polyoxyethylenated, and then preferably comprising from 1 to 50 ethylene oxide units and better still from 2 to 10 ethylene oxide units.

Preferentially, the sulfonate anionic surfactants are selected, alone or as a combination, from:
C6-C24 and especially C12-C20 alkylsulfosuccinates, especially laurylsulfosuccinates;
C6-C24 and especially C12-C20 alkyl ether sulfosuccinates;
C6-C24 acylisethionates and preferably C12-C18 acylisethionates, in particular in the form of alkali metal or alkaline-earth metal, ammonium or amino alcohol salts.

The sulfate anionic surfactants that may be used comprise at least one sulfate function (—OSO₃H or —OSO₃).

They may be selected from the following compounds: alkyl sulfates, alkyl ether sulfates, alkylamido ether sulfates, alkylaryl polyether sulfates, monoglyceride sulfates; and also the salts of these compounds;

the alkyl groups of these compounds comprising from 6 to 30 carbon atoms, especially from 12 to 28, better still from 14 to 24 or even from 16 to 22 carbon atoms;

the aryl group preferably denoting a phenyl or benzyl group;

these compounds possibly being polyoxyalkylenated, especially polyoxyethylenated, and then preferably comprising from 1 to 50 ethylene oxide units and better still from 2 to 10 ethylene oxide units.

Preferentially, the sulfate anionic surfactants are selected, alone or as a combination, from:
C6-C24 alkyl sulfates and especially C12-C20 alkyl sulfates,
alkyl ether sulfates, especially of C6-C24 or even C12-C20, preferably comprising from 2 to 20 ethylene oxide units;
in particular in the form of alkali metal or alkaline-earth metal, ammonium or amino alcohol salts.

When the anionic surfactant is in salt form, the said salt may be selected from alkali metal salts, such as the sodium or potassium salt, ammonium salts, amine salts and in particular amino alcohol salts, and alkaline-earth metal salts, such as the magnesium salt. Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl) aminomethane salts.

Alkali metal or alkaline-earth metal salts and in particular the sodium or magnesium salts are preferably used.

Preferentially, the anionic surfactants are selected, alone or as a combination, from:
C6-C24 and especially C12-C20 alkyl sulfates;
C6-C24 and especially C12-C20 alkyl ether sulfates; preferably comprising from 2 to 20 ethylene oxide units;
C6-C24 and especially C12-C20 alkylsulfosuccinates, especially laurylsulfosuccinates;
C6-C24 and especially C12-C20 alkyl ether sulfosuccinates;
C6-C24 acylisethionates and preferably C12-C18 acylisethionates;
in particular in the form of alkali metal such as sodium or alkaline-earth metal, ammonium or amino alcohol salts.

Preferably, the anionic surfactant is selected from sodium laureth sulfate, sodium lauryl sulfate, and a combination thereof.

Advantageously, the anionic surfactant is present in an amount ranging from 5 wt. % to 50 wt. %, preferably from 9 wt. % to 25 wt. %, more preferably from 12 wt. % to 18 wt. %, relative to the total weight of the composition.

### Hydroxy Acids

The composition according to the present invention comprises at least one alpha hydroxy acid and at least one beta hydroxy acid.

### Alpha Hydroxy Acids

The term “alpha-hydroxy acid” is understood to mean, according to the present invention, a carboxylic acid having at least one hydroxyl functional group occupying an alpha-position on said acid (carbon adjacent to a carboxylic acid functional group). Suitable alpha hydroxy acids includes glycolic acid, citric acid, lactic acid, methyllactic acid, glucuronic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, tartaric acid, malic acid, fumaric acid.

Preferably, the alpha hydroxy acid is selected from lactic acid, glycolic acid, tartaric acid, mandelic acid, citric acid, and combinations thereof.

More preferably, the alpha hydroxy acid in the composition consists of lactic acid.

Lactic acid, or 2-hydroxypropanoic acid, provides soft peeling on excess sebum, residues and loose dead cells around follicles and makes hair roots energized and lifted. In addition, lactic acid also boosts production of glycosaminoglycan (GAG) in the skin, improving the barrier function and moisturization of skin.

Advantageously, the alpha hydroxy acid is present in an amount ranging from 0.3 wt. % to 30 wt. %, preferably from 1.5 wt. % to 15 wt. %, and more preferably from 2 wt. % to 8 wt. %, relative to the total weight of the composition.

### Beta Hydroxy Acids

The term “beta-hydroxy acid” is understood to mean, according to the present invention, a carboxylic acid having a hydroxyl functional group and a carboxylic functional group separated by two carbon atoms.

Suitable beta hydroxy acids includes salicylic acid, propionic acid, beta-hydroybutyric acid, beta-hydroxy beata-methylbutyric acid, carnitine, derivatives thereof, and combinations thereof.

Preferably, the beta hydroxy acid is selected from salicylic acid and its derivative of the formula:

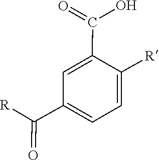

wherein R is a linear, branched or cyclic saturated aliphatic group or an aliphatic unsaturated group containing one or a number of double bonds, which may or may not be conjugated, these groups containing from 2 to 22, preferably 3 to 11 carbon atoms and being able to be substituted for example by at least one substituent selected from (a) halogen atoms, (b) the trifluoromethyl group, (c) hydroxyl groups in the free form or esterified by an acid having from 1 to 6 carbon atoms or (d) a carboxyl functional group which is free or esterified by a lower alcohol having from 1 to 6 carbon atoms;

R is a hydroxyl group or an ester functional group of the following formula:

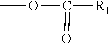

wherein R₁ is a linear or branched saturated or unsaturated aliphatic group having from 1 to 18 carbon atoms.

Preferred salicylic acid derivatives include 5-n-octanoyl salicylic acid (capryloyl salicylic acid), 5-n-decanoyl salicylic acid, 5-n-dodecanoyl salicylic acid, 5-n-heptyloxy salicylic acid and 4-n-heptyloxy salicylic acid.

More preferably, the beta hydroxy acid is selected from salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combination thereof.

Most preferably, the beta hydroxy acid is salicylic acid.

Salicylic acid, or 2-hydroxybenzoic acid, is provided to the chemical peel composition to provide enhanced penetration of the composition into the skin. Salicylic acid penetrates deeper into the skin than alpha hydroxy acids, such as lactic acid.

Advantageously, the beta-hydroxy is present in an amount ranging from 0.3 wt. % to 10 wt. %, preferably from 0.5 wt. % to 5 wt. %, and more preferably from 1 wt. % to 4 wt. %, relative to the total weight of the composition.

The inventors have found that the composition according to the present invention can remove the stubborn sebum effectively. When both alpha hydroxy acid and beta hydroxy acid, especially lactic acid and salicylic acid or its derivative, lactic acid can breakdown the desmosomes protein and peel off the dead cells, while salicylic acid or its derivative can good deep into the follicles loosen the stubborn sebum in it. Thus, the combination of surfactant and hydroxy acids deliver a long-lasting clean effect.

### Cationic Polymers

Preferably, the composition according to the present invention further comprises at least one cationic polymer.

Preferably, the cationic polymer is selected from cationic guar compounds.

Suitable examples of cationic guar compounds are non-cellulose cationic polysaccharides, such as guar gums containing trialkylammonium cationic groups, for example hydroxy C1-C4 alkyl guar hydroxy C1-C4 alkyltrimonium chloride, guar hydroxy C1-C4 alkyl trimonium chloride.

Suitable cationic guar gum derivatives are those given the PCPC (Personal Care Products Council, formerly CTFA, designation) of guar hydroxypropyl trimonium chloride, available commercially for example as JAGUAR C13S, which has a low degree of substitution of the cationic groups and a high viscosity. The low degree of cationic substitution leads to a cationic charge density of 0.0008. Other suitable materials include that known as JAGUAR C15, having a moderate degree of substitution and a low viscosity, JAGUAR C17 (high degree of substitution, hence cationic charge density of 0.0016, high viscosity) and JAGUAR C16 which is a hydroxypropylated cationic guar derivative containing a low level of substituent groups as well as cationic quaternary ammonium groups. JAGUAR C16 has a cationic charge density of 0.0008.

Guar hydroxypropyl trimonium chloride, may also be available commercially for example as N-HANCE CG13 from the company Ashland.

Also suitable is hydroxypropyl guar hydroxypropyltrimonium chloride, commercially available as JAGUAR 162, which is a high transparency, medium viscosity guar having a low degree of substitution.

Preferably, the cationic polymer is selected from hydroxy C1-C4 alkyl guar hydroxy C1-C4 alkyltrimonium chloride, guar hydroxy C1-C4 alkyl trimonium chloride, and a combination thereof.

More preferably, the cationic polymer is selected from hydroxypropyl guar hydroxypropyltrimonium chloride, guar hydroxypropyltrimonium chloride, and a combination thereof.

Most preferably, the cationic polymer is hydroxypropyl guar hydroxypropyltrimonium chloride.

Preferably, if presents, the cationic polymer is present in an amount ranging from 0.01 wt. % to 10 wt. %, preferably from 0.03 wt. % to 5 wt. %, more preferably from 0.1 wt. % to 1 wt. %, relative to the total weight of the composition.

### Alkanolamines

Preferably, the composition according to the invention additionally comprises at least one alkanolamine.

The term “alkanolamine” is understood to mean, within the meaning of the invention, an organic amine comprising a primary, secondary or tertiary amine functional group, and one or more linear or branched C1 to C8 alkyl groups carrying one or more hydroxyl radicals.

Alkanolamines, such as monoalkanolamines, dialkanolamines or trialkanolamines, comprising from one to three, identical or different, C1 to C4 hydroxyalkyl radicals are in particular suitable for the implementation of the invention.

Mention may be made, among the alkanolamines, of monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethyl) aminomethane, and their combinations.

Preferably, the composition according to the invention comprises at least one alkanolamine selected from monoethanolamine, diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethyl) aminomethane, and a combination thereof.

More preferably, the alkanolamine is selected from monoethanolamine, diethanolamine, triethanolamine, N,N-dimethylethanolamine, and a combination thereof.

Most preferably, the composition according the present invention comprise triethanolamine as alkanolamine.

It has been found that high amount of acid brings an issue that cationic polymer cannot attach on hair fiber surface because electrostatic screening. The alkanolamine can react with the hydroxy acid to form a cleating-like molecular to decrease electrostatic screening so that the cationic polymer deposition is improved.

Preferably, if presents, the alkanolamine is present in an amount ranging from 0.1 wt. % to 5 wt. %, preferably from 1 wt. % to 4 wt. %, and more preferably from 1.5 wt. % to 3 wt. %, relative to the total weight of the composition.

The inventors have found that with the presence of the cationic polymer and the alkanolamine, the composition according to the present invention can demonstrate a good conditioning effect on the hair, for example, the smoothness of the hair at wet state and/or dry state is improved.

As used herein, “conditioning” means imparting to the hair at least one property selected from compatibility, manageability, moisture-retentivity, luster, shine, smoothness and softness. The state of conditioning is evaluated by comparing the hair treated with the composition to be tested in contrast with the hair treated with a commercial product.

### Aqueous Phase

The composition according to the present invention comprises an aqueous phase.

Preferably, the aqueous phase is a continuous phase.

The aqueous phase of the present invention comprises water.

The aqueous phase may also comprise water-miscible organic solvents (at room temperature of 20-25° C.), for instance polyols such as C2-C₆ polyols, more particularly glycerin, hexylene glycol; glycol ethers (especially containing from 3 to 16 carbon atoms) such as mono-, di- or tripropylene glycol (C1-C₄)alkyl ethers, mono-, di- or triethylene glycol (C1-C₄)alkyl ethers, and combinations thereof.

Advantageously, the aqueous phase is present in an amount ranging from 70 wt. % to 90 wt. %, preferably from 70 wt. % to 80 wt. %, relative to the total weight of the composition.

### Additional Ingredients

The composition according to the present invention may also comprise any other additional ingredient that is usually used in the field of self-cleaning products, in particular shampoos.

The skilled in the art can select the amount of the additional ingredients so as not to adversely impact the final use of the composition according to the present invention.

For example, such additional ingredients include pH adjusting agents, preserving agents, antioxidants, fragrances, electrolytes and stabilizers, plant extracts, proteins, amino acids, vitamins, glycols, emollients, derivatives of the foregoing, and combinations thereof.

Non-limiting examples of pH adjusting agents includes potassium acetate, potassium hydroxide, sodium carbonate, sodium hydroxide, phosphoric acid, succinic acid, sodium citrate, sodium hydrogen carbonate, and combinations thereof.

The pH adjusting agent is used in an amount so that the pH of the composition of the present invention is less than or equal to 6.5, preferably from 2 to 6.5, more preferably from 3.5 to 4.5.

According to a preferred embodiment, the present invention provides a composition for cleansing the hair comprising, relative to the total weight of the composition:
a) from 12 wt. % to 18 wt. % of at least one anionic surfactant;
b) from 2 wt. % to 8 wt. % of lactic acid and from 1 wt. % to 4 wt. % of at least one beta hydroxy acid selected from salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combination thereof;
c) from 0.1 wt. % to 1 wt. % of at least one cationic polymer selected from hydroxypropyl guar hydroxypropyltrimonium chloride, guar hydroxypropyltrimonium chloride, and a combination thereof; and
d) from 1.5 wt. % to 3 wt. % of at least one alkanolamine selected from monoethanolamine, diethanolamine, triethanolamine, N,N-dimethylethanolamine, and a combination thereof,
wherein the pH of the composition is from 3.5 to 4.5.

### Preparation and Use

The composition according to the present invention can be prepared by mixing ingredients a) to d), as essential ingredients, as well as additional ingredient(s), as explained above.

The method and means to mix the above essential and optional ingredients are not limited. Any conventional method and means can be used to mix the above essential and optional ingredients to prepare the composition according to the present invention.

The composition according to the present invention can have a gel-like texture. According to the second aspect of the present invention, a process for cleansing the hair comprising applying the composition as described above onto the hair, and then rinsing with water after an optional period of exposure.

The following examples are given by way of illustration of the present invention and shall not be interpreted as limiting the scope.

### EXAMPLES

Main raw materials used, trade names and supplier thereof are listed in Table 1.

TABLE 1

| INCI Name | Trade Name | Supplier |
|---|---|---|
| Lactic acid | L(+)-LACTIC ACID 90% HEAT | JUNGBUNZLAUER |
| | STABLE PERSONAL CARE | |
| | GRADE | |
| Salicylic acid | SALICYLIC ACID USP | ALTA LABORATORIES |
| Triethanolamine | TRIETANOLAMINA 99 | OXITENO |
| Hydroxypropyl guar | JAGUAR C 162 SGI | RHODIA (SOLVAY) |
| hydroxypropyltrimonium | | |
| chloride | | |
| Sodium laureth sulfate | GALAXY LES 170-MB | GALAXY SURFACTANTS |

### Invention Examples 1-4 and Comparative Examples 1-4

Shampoos according to invention example s (IE.) 1-4 and comparative examples (CE.) 1-4 were prepared with the ingredients listed in Table 2 (the contents are expressed as weight percentages of active material with regard to the total weight of each shampoo, unless otherwise indicated):

TABLE 2

| Ingredients | IE. 1 | IE. 2 | IE. 3 | IE. 4 | CE. 1 | CE. 2 | CE. 3 | CE. 4 |
|---|---|---|---|---|---|---|---|---|
| Sodium laureth sulfate | 15 | 15 | 15 | 15 | 15 | 15 | 15 | 15 |
| Lactic acid | 6 | 6 | 6 | 6 | — | 0.20 | 7.5 | — |
| Salicylic acid | 1.5 | 1.5 | 1.5 | 1.5 | 0.2 | — | — | 3.5 |
| Triethanolamine | 2.2 | 1.1 | — | 2.2 | — | — | 2.2 | 2.2 |
| Hydroxypropyl guar | 0.35 | 0.35 | 0.35 | — | 0.35 | 0.35 | 0.35 | 0.35 |
| hydroxypropyltrimonium | | | | | | | | |
| chloride | | | | | | | | |
| Sodium Hydroxide | 0.38 | 0.38 | 1.27 | 1.27 | 0.14 | 0.10 | 1.52 | 0.76 |
| water | 74.6 | 75.7 | 75.9 | 74.0 | 84.3 | 84.4 | 74.43 | 78.19 |
| pH | 3.8 | 3.8 | 3.8 | 3.8 | 4.3 | 4.5 | 3.8 | 3.8 |

Shampoos of invention examples (IE.) 1-4 are compositions according to the present invention.

Shampoo of comparative example (CE.) 1 comprises no alpha hydroxy acid.

Shampoo of comparative example (CE.) 2 comprises no beta hydroxy acid.

Shampoo of comparative example (CE.) 3 comprises no beta hydroxy acid.

Shampoo of comparative example (CE.) 4 comprises no alpha hydroxy acid.

### Preparation Procedure:

Each shampoo was prepared as follows, taking shampoo of invention example 1 for illustration.
1) adding hydroxypropyl guar hydroxypropyltrimonium chloride into water with stirring to form a transparent base at room temperature;
2) adding sodium laureth sulfate into the transparent base with stirring;
3) adding lactic acid, salicylic acid, triethanolamine, and sodium hydroxide to obtain the shampoo.

### Evaluation:

The conditioning effect of each shampoo was evaluated as follows:

I) Treating the Hair with the Tested Shampoo:

A bundle of damaged hair was selected. The hair was combed and wet under running water, the tested shampoo was applied uniformly on the damaged hair from root to tail, the shampoo was gently massaged into the hair with two fingers from root to tail (without knotting) to foam the shampoo. The shampoo was rinsed off under a tap water until all foam was washed off.

### II) Rinse Smoothness:

The hair was stroked by two fingers from the root during rinse, the degree of non-resistance was judged when finger was sliding through the hair.

### III) Evaluating Smoothness and Softness in the Wet State:

The hair was stroked by two fingers from the root, and the haircare performance of the shampoo was judged according to the smoothness of the surface of the hair.

The hair was curled from the tail by using two fingers, and the softness of the hair were judged on the hair surface according to whether the sample is easy to curl and the force generated in the curling process.

### IV) Evaluating Smoothness and Softness in the Dry State and Dry Hair Coating:

The hair were either air dried naturally or blown in a 60° C. oven to a completely dry condition.

The hair was stroked by two fingers from the root, and the hair care performance of the shampoo was judged according to the smoothness of the surface of the hair.

The hair was stroked by two fingers from the root, and the intensity of deposit and residue on hair was judged.

The hair was curled from the tail by using two fingers, and the softness of the hair were judged on the hair surface according to whether the sample is easy to curl and the force generated in the curling process.

A non-silicone shampoo (Loreal Hyaluronic Acid Shampoo-WATER PUMP from Loreal) was used as the benchmark in parallel with the tested shampoo in above conditioning effect evaluation.

The cleansing effect of each shampoo was evaluated as follows:

6 volunteers were recruited, one half of the hair of each volunteer was washed with the shampoo to be tested, and the other half of the hair of each volunteer was washed with a benchmark.

A non-silicone shampoo (Loreal Hyaluronic Acid Shampoo-WATER PUMP from Loreal) was used as the benchmark in above cleansing effect evaluation.

The scores on cleansing effect of each tested shampoo were given by volunteers 24 hours after application and averaged.

The scores on conditioning effect and cleansing effect of each tested shampoo were given by volunteers and averaged, wherein the scores of cleansing effect were given 24 hours after application.

The scoring standard for conditioning effect and cleansing effect of each tested shampoo is as follows.
--: the tested shampoo is significantly inferior to the benchmark;
-: the tested shampoo is slightly inferior to the benchmark;
=: the tested shampoo is similar to the benchmark;
+: the tested shampoo is slightly better than the benchmark; and
++: the tested shampoo is obviously superior to the benchmark.
The results on conditioning effect and cleansing effect were summarized in Table 3.

TABLE 3

| Properties | IE. 1 | IE. 2 | IE. 3 | IE. 4 | CE. 1 | CE. 2 | CE. 3 | CE. 4 |
|---|---|---|---|---|---|---|---|---|
| Rinse smoothness | ++ | + | −− | −− | = | = | + | + |
| Wet hair smoothness | ++ | + | − | −− | = | = | + | − |
| Wet hair softness | + | + | − | − | = | = | + | + |
| Dry hair smoothness | + | + | − | −− | = | = | + | + |
| Dry hair softness | + | + | − | − | = | = | + | + |
| Dry hair coating | ++ | + | − | −− | = | = | = | = |
| Long lasting clean scalp | + | + | + | + | − | − | = | − |

It can be seen that the composition according to the present invention can provide a long-lasting clean effect.

It can be also seen that with the presence of the cationic polymer and the alkanolamine, the composition according to the present invention can demonstrate a good conditioning effect on the hair, for example, the smoothness and softness of the hair at wet state and dry state is improved.

1. A composition for cleansing the hair comprising:
a) at least one anionic surfactant;
b) at least one alpha hydroxy acid; and
c) at least one beta hydroxy acid.

2. The composition according to claim 1, which is silicon free.

3. The composition according to claim 1, wherein the anionic surfactant is selected from the group consisting of:
C6-C24 alkyl sulfates;
C6-C24 alkyl ether sulfates;
C6-C24 alkylsulfosuccinates,
C6-C24 alkyl ether sulfosuccinates; and
C6-C24 acylisethionates;
in the form of alkali metal, alkaline-earth metal, ammonium or amino alcohol salts.

4. The composition according to claim 1, wherein the anionic surfactant is present in an amount ranging from 5 wt. % to 50 wt. %, relative to the total weight of the composition.

5. The composition according to claim 1, wherein the alpha hydroxy acid is selected from the group consisting of lactic acid, glycolic acid, tartaric acid, mandelic acid, citric acid, and combinations thereof.

6. The composition according to claim 1, wherein the alpha hydroxy acid is present in an amount ranging from 0.3 wt. % to 30 wt. %, wt. %, relative to the total weight of the composition.

7. The composition according to claim 1, wherein the beta hydroxy acid is selected from salicylic acid and its derivative of the formula: 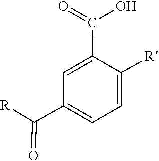
wherein R is a linear, branched or cyclic saturated aliphatic group or an aliphatic unsaturated group containing one or a number of double bonds, which may or may not be conjugated, these groups containing from 2 to 22, carbon atoms optionally substituted by at least one substituent selected from the group consisting of (a) halogen atoms, (b) the trifluoromethyl group, (c) hydroxyl groups in the free form or esterified by an acid having from 1 to 6 carbon atoms [or] and (d) a carboxyl functional group which is free or esterified by a lower alcohol having from 1 to 6 carbon atoms;
R′ is a hydroxyl group or an ester functional group of the following formula: 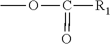
wherein R¹ is a linear or branched saturated or unsaturated aliphatic group having from 1 to 18 carbon atoms.

8. The composition according to claim 1, wherein the beta hydroxy acid is present in an amount ranging from 0.3 wt. % to 10 wt. %, relative to the total weight of the composition.

9. The composition according to claim 1, further comprising at least one cationic polymer.

10. The composition according to claim 9, wherein the cationic polymer is present in an amount ranging from 0.01 wt. % to 10 wt. %, relative to the total weight of the composition.

11. The composition according to claim 1, comprising at least one alkanolamine.

12. The composition according to claim 11, wherein the alkanolamine is present in an amount ranging from 0.1 wt. % to 5 wt. %, relative to the total weight of the composition.

13. The composition according to claim 1, wherein the pH of the composition is from 2 to 6.5.

14. The composition according to claim 1, comprising, relative to the total weight of the composition:
a) from 12 wt. % to 18 wt. % of at least one anionic surfactant;
b) from 2 wt. % to 8 wt. % of lactic acid and from 1 wt. % to 4 wt. % of at least one beta hydroxy acid selected from salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combination thereof;
c) from 0.1 wt. % to 1 wt. % of at least one cationic polymer selected from hydroxypropyl guar hydroxypropyltrimonium chloride, guar hydroxypropyltrimonium chloride, and a combination thereof; and
d) from 1.5 wt. % to 3 wt. % of at least one alkanolamine selected from monoethanolamine, diethanolamine, triethanolamine, N,N-dimethylethanolamine, and a combination thereof,
wherein the pH of the composition is from 3.5 to 4.5.

15. A process for cleansing the hair comprising applying the composition according to claim 1 onto the hair, and then rinsing the hair with water after a period of exposure.

16. The composition according to claim 9, wherein the alkanolamine is selected from the group consisting of monoethanolamine, diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethyl) aminomethane, and a combination thereof.

17. The composition according to claim 11, wherein the, the alkanolamine is selected from the group consisting of monoethanolamine, diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethyl) aminomethane, and a combination thereof.
